(19) United States
(12) Patent Application Publication
(10) Pub. No.: US 2009/0317830 A1
(43) Pub. Date: Dec. 24, 2009

(54) HEPATITIS C VIRUS NS2/3 ASSAY

(75) Inventors: Lyne LAMARRE, Blainville (CA); Lisette LAGACE, Lorraine (CA); Diane THIBEAULT, Laval (CA)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/545,833

(22) Filed: Aug. 23, 2009

Related U.S. Application Data
(63) Continuation of application No. 11/275,284, filed on Dec. 21, 2005, now abandoned.

(51) Int. Cl. G01N 33/573; C07K 16/00

(52) U.S. Cl. 435/7.4; 530/387.9

(57) ABSTRACT

The present invention provides an assay for the detection of the NS2/3 cleavage products NS2 or NS3 in the presence of uncleaved NS2/3. Following self-cleavage of NS2/3 to generate NS2 and NS3 cleavage products, a sample is incubated with a ligand specific for the recognition of NS2 or NS3 cleavage product in the presence of uncleaved NS2/3. There is provided a method for detecting a NS2/3 autocleavage product in a sample containing NS2/3 protease, whereby the amount of bound ligand detected correlates with the NS2/3 autocleavage activity. A further aspect of the present invention concerns ligands selectively recognizing one of the NS2 cleavage product or the NS3 cleavage product with minimal cross-reactivity with the uncleaved NS2/3 and the other cleaved product. The present invention provides antibodies that selectively recognize one of cleaved NS2 product or cleaved NS3 product with minimal cross-reactivity with the uncleaved NS2/3 and the other cleaved product.

### APPLICATION DATA

This application is a continuation of U.S. Ser. No. 11/275,284 filed Dec. 21, 2005.

### FIELD OF THE INVENTION

The present invention relates to an assay for detecting cleavage of HCV protein in a sample, and more particularly, to an assay for the selective detection of HCV NS2/3 autocleavage activity, and even more particularly to the identification of potential HCV inhibitor compounds.

### BACKGROUND OF THE INVENTION

Hepatitis C virus (HCV) is the major etiological agent of post-transfusion and community-acquired, non-A, non-B hepatitis worldwide. A high percentage of carriers become chronically infected and many progress to chronic liver disease, so called chronic hepatitis C. This group is in turn at high risk for serious liver disease such as liver cirrhosis, hepatocellular carcinoma, and terminal liver disease leading to death.

HCV is an enveloped positive strand RNA virus in the Flaviviridae family. The single strand HCV RNA genome is of positive polarity and comprises one open reading frame (ORF) of approximately 9600 nucleotides in length, which encodes a linear polyprotein of approx. 3010 amino acids. In infected cells, this polyprotein is cleaved at multiple sites by cellular and viral proteases to produce structural and non-structural (NS) proteins. The structural proteins (C, E1, E2 and p7) comprise polypeptides that constitute the viral particle. Processing of the structural proteins is catalyzed by host cell proteases. The non-structural proteins (NS2, NS3, NS4A, NS4B, NS5A, NS5B) encode for enzymes or accessory factors that catalyze and regulate the replication of the HCV RNA genome. The generation of the mature non-structural proteins is catalyzed by two virally encoded proteases. The first is the NS2/3 protease which auto-catalyses the cleavage between NS2 and NS3. The NS3 contains a N-terminal serine protease domain and catalyzes the remaining cleavages from the polyprotein. The released NS4A protein has at least two roles. The first role is forming a stable complex with NS3 protein and assisting in the membrane localization of the NS3/NS4A complex; the second is acting as a cofactor for NS3 protease activity. This membrane-associated complex, in turn catalyzes the cleavage of the remaining sites on the polyprotein, thus effecting the release of NS4B, NS5A and NS5B.

The cleavage of the Hepatitis C Virus (HCV) polyprotein between the nonstructural proteins NS2 and NS3 is mediated by the NS2/3 protease, a protease activity that is encoded by the NS2 region and the minimal NS3 protease domain which flank the cleavage site. NS2/3 protease is expressed in virally infected hepatocytes and experimental data are consistent with its essential role in viral propagation and disease. Indeed, no productive infection was observed in chimpanzees upon inoculation of HCV clones containing mutations abolishing NS2/3 protease activity, suggesting that this HCV-encoded enzyme is essential for productive replication in vivo (1).

A minimal catalytic region of NS2/3 protease has been defined and includes the C-terminus of NS2 and the N-terminal NS3 protease domain (2-5). The NS2/3 (904-1206) variant from HCV genotype 1b was purified from E. coli inclusion bodies and refolded by gel filtration chromatography as previously described (2, 3). The purified inactive form of NS2/3 (904-1206) can be activated by the addition of glycerol and detergent to induce autocleavage at the predicted site between the residues leucine 1026 and alanine 1027 (2, 3).

NS2/3 protease cleavage detection assays based on the separation of the NS2 and NS3 products from the NS2/3 precursor by SDS-PAGE and by HPLC have been reported, as well as an assay based on the NS3 protease activity of the NS2/3 protein which also requires separation of the NS2/3 uncleaved precursor from the NS3 protease product (2-5). Such methods can be time-consuming and are not adapted for rapid screening. Moreover, no assay has yet been developed having the selectivity to detect NS2/3 cleavage products in the presence of uncleaved NS2/3.

It would, thus, be desirable to develop efficient NS2/3 cleavage assays which overcome one or more disadvantages of existing assays.

Novel selective assay methods are provided comprising cleavage of NS2/3 protease in a sample and treatment of the cleaved sample which enables detection of cleavage products NS2 or NS3 therein.

### SUMMARY OF THE INVENTION

The present invention provides a novel assay for NS2/3 cleavage detection. More particularly, the present invention provides a novel assay for the detection of the NS2/3 cleavage products NS2 or NS3 in the presence of uncleaved NS2/3.

In the present invention, following self-cleavage of NS2/3 to generate NS2 and NS3 cleavage products, the sample is incubated with a ligand specific for the recognition of NS2 or NS3 cleavage product in the presence of uncleaved NS2/3.

In a first aspect of the present invention, there is provided a method for detecting a NS2/3 autocleavage product in a sample containing refolded, inactive NS2/3 protease, the method comprising:
a) subjecting the sample to suitable conditions under which at least a portion of the NS2/3 protease is self-cleaved to yield cleaved NS2 and NS3 products;
b) incubating the NS2 and NS3 products with a ligand that can preferentially bind to one of either NS2 or NS3 product over NS2/3 under conditions suitable to afford binding of the ligand to NS2 or NS3; and
c) detecting NS2- or NS3-bound ligand produced in step b), whereby the amount of bound ligand detected correlates with the NS2/3 autocleavage activity.

The present method is also useful as an assay to screen candidate NS2/3 inhibitor compounds.

A second aspect provides for an assay for screening a candidate compound for NS2/3 cleavage inhibitory activity in a sample containing refolded, inactive NS2/3 protease, the assay comprising:
a) subjecting a first sample comprising NS2/3 protease to suitable conditions under which at least a portion of NS2/3 is self-cleaved to yield cleaved NS2 and NS3 products in the absence of a candidate compound;
b) subjecting a second sample comprising NS2/3 protease in the presence of a candidate compound under the same conditions as those in step a);
c) incubating the first and second samples with a ligand that preferentially binds to one of either NS2 or NS3 product over NS2/3 under conditions suitable to cause binding of the ligand to NS2 or NS3; and
d) determining the amount of ligand bound to the second sample and to the first sample produced in step c), whereby a decrease in the amount of ligand in the second sample compared to that of the first sample indicates that the candidate compound may be an inhibitor of NS2/3 autocleavage activity.

A further aspect of the present invention concerns ligands selectively recognizing one of the NS2 cleaved product or the NS3 cleaved product with minimal cross-reactivity with the uncleaved NS2/3 and the other cleaved product. Specifically, the present invention provides antibodies that selectively recognize cleaved NS3 product with minimal cross-reactivity with the uncleaved NS2/3 and the NS2 cleaved product. Alternatively, the present invention provides antibodies that selectively recognize cleaved NS2 product with minimal cross-reactivity with the uncleaved NS2/3 and the NS3 cleaved product.

As will be recognized by persons of skill in the art, other types of auto-cleaving proteases similar or homologous to the HCV NS2/3 protease may be used in the method/assay of the present invention in the search for respective inhibitors. Such other proteases may be found in pestiviruses, such as, but not limited to: GB virus A, B, or C; bovine viral diarrhea virus (BVDV); Classical Swine Fever virus; Border disease virus; bovine pestivirus; and porcine pestivirus.

These and other aspects of the present invention are described herein by reference to the following figures.

### BRIEF DESCRIPTION OF THE DRAWINGS

FIG. 1A is a schematic representation of an NS2/3 protease assay in accordance with one aspect of the present invention;

FIG. 1B is a schematic representation of an NS2/3 protease assay in accordance with another aspect of the present invention;

FIG. 2A illustrates the dilution curve of the K147 polyclonal antibody as determined by ELISA assay;

FIG. 2B is a Western blot demonstrating the selective binding of K147 antibody to (+) cleaved NS3 product compared to (−) uncleaved NS2/3;

FIG. 3 graphically illustrates the correlation between binding of K147 antibody to the autocleavage activity of the NS2/3 protease according to the method of Example 2;

FIG. 4 graphically illustrates the concentration of NS2/3 protease in the cleavage step according to the method of Example 3;

FIG. 5 graphically illustrates the time dependence of NS2/3 protease cleavage step according to the method of Example 3;

FIG. 6 graphically illustrates the titration of the NS3-selective rabbit polyclonal antibody K147;

FIG. 7A is a schematic representation of an NS2/3 protease assay in accordance with a specific embodiment of the present invention;

FIG. 7B is a schematic representation of an NS2/3 protease assay in accordance with another embodiment of the present invention;

FIG. 8 graphically illustrates the Z′ obtained with an embodiment of the present NS2/3 protease assay in accordance with Example 5; and

FIG. 9 graphically illustrates an IC₅₀ curve of compound A obtained with the NS2/3 protease assay in accordance with Example 5.

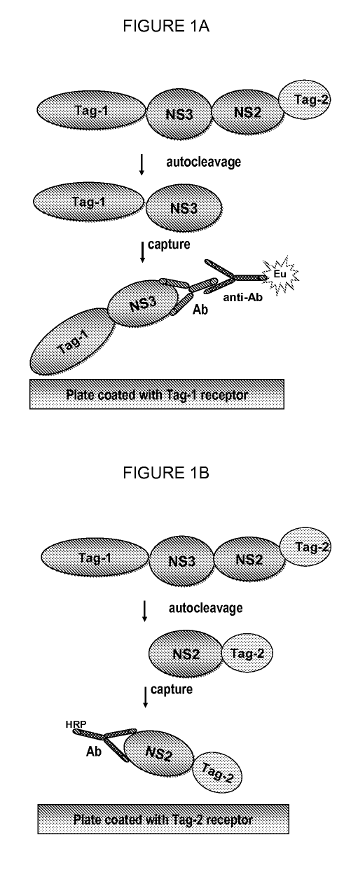

### DETAILED DESCRIPTION OF THE PRESENT INVENTION

### Definitions

Unless otherwise defined, all technical and scientific terms used herein have the same meaning as those commonly understood by one of ordinary skill in the art to which the invention pertains. Generally, the procedures for cell culture, infection, protein purification, molecular biology methods, and the like are common methods used in the art. Such techniques can be found in reference manuals such as, for example, Sambrook et al. (2001, Molecular Cloning—A Laboratory Manual, Cold Spring Harbor Laboratory Press); Ausubel et al. (1994, Current Protocols in Molecular Biology, Wiley, New York) and Coligan et al. (1995, Current Protocols in Protein Science, Volume 1, John Wiley & Sons, Inc., New York).

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission (Biochemistry, 1972, 11:1726-1732).

All values and concentrations presented herein are subject to inherent variations acceptable in biological science within an error of ±10%. The term “about” also refers to this acceptable variation.

### NS2/3 Protease

The term “NS2/3”, “NS2/3 protein”, “NS2/3 protease” or “uncleaved NS2/3 protease”, used herein interchangeably, refer to the region of the Hepatitis C Virus (HCV all genotypes) polyprotein that catalyzes the cleavage of the NS2 domain (810-1026) from the NS3 domain (1027-1615), as well as functionally equivalent variants thereof. In one embodiment as described herein, it is encoded by the native NS2 region (specifically, amino acids 810 to 1026) and the minimal NS3 protease domain (1027 to 1206) of the polyprotein (numbered according to genotype 1a H77 sequence, GenBank accession number AAB67036) herein referred to as 810*-1206 [SEQ ID NO.1; *where amino acid 810 corresponds to amino acid 1 of SEQ ID NO.1].

Functionally equivalent variants of the NS2/3 protease are encompassed by the term “NS2/3,” “NS2/3 protein,” “NS2/3 protease,” or “uncleaved NS2/3,” such functionally equivalent referring to variants able to catalyze the cleavage of NS2/3 such as variants from other HCV isolates/genotypes. The term “variant” also refers to a protein derived from native NS2/3, but modified in sequence by insertion, deletion, substitution, or modification of one or more amino acids. With respect to amino acid substitutions, these will generally include conservative amino acid substitutions that do not affect the NS2/3 function of the protein as would be appreciated by one of skill in the art. It also includes modified amino acids, for example, amino acids including modified side chains.

Furthermore, a “functionally equivalent variant” refers to truncations comprising the minimal catalytic region of the NS2/3 protease that has been determined to comprise the C-terminus of NS2 (beginning at about amino acid position 907 of the polyprotein) and the N-terminus of NS3 (up to amino acid position 1206) (5). Accordingly, NS2/3 truncations comprising these amino acid deletions, termed “NS2/3 fragment” are examples of variants in accordance with the present invention, such as: (907-1206; SEQ ID NO. 2) or (904-1206; SEQ ID NO. 3). Additionally, NS2/3 deletion mutants comprising any number of amino acid deletions between the native sequence of NS2/3 (810-1615 or 810-1206) and truncated NS2/3 (907-1206) are also contemplated to be variants in accordance with the present invention. Other variants are likewise known in the art, such as those described in WO 01/68818 (5), WO 02/48375 & U.S. Pat. No. 6,815,159 (2).

As is well recognized within the skill or the art, the term “variant” also encompasses modifications to the protein such as adding affinity tags or detectable labels in order to facilitate extraction/purification or detection/measurement. Also, substitutions or insertions, such as addition of amino acid(s) to enhance solubility (such as lysine), are also encompassed with the term “variant.” One example of such variant is (Lys₄-His₆-904-1206-StrepTag-Lys₄) [SEQ ID NO. 4].

If a NS2/3 protease functionally equivalent variant is used in the assay in accordance with the present invention, it is necessary to confirm that the modified protein retains NS2/3 autocleavage activity. This can be done using standard cleavage assays such as those described in references 2-5, cited herein.

The term “at least a portion of NS2/3 protease is cleaved” means that at least a portion of the total amount of the NS2/3 protease present in the assay mixture is cleaved at the 1026-1027 cleavage site.

### NS2 Product

As used herein, the term “NS2 product” refers to NS2 domain that is cleaved or released from the NS2/3 protease. NS2 product may correspond with native NS2, or may be a functionally equivalent variant thereof. In one embodiment in a construct described herein, native NS2 is represented by amino acids 810-1026 [1-217 of SEQ. ID. No. 1]; however, one of skill in the art will appreciate that NS2 product in accordance with the present invention may be modified by insertion, deletion, modification, substitution of one or more amino acids as described above. It is anticipated that such modifications will correspond with modifications existing in the NS2 portion of the NS2/3 protease utilized in the assay. The term “NS2 product” is interchangeably used herein with the terms “NS2” or “cleaved NS2 product.”

Accordingly, NS2 truncations comprising amino acid deletions, termed “NS2 fragment,” such as fragments 907-1026 from SEQ ID NO. 2 or 904-1026 from SEQ ID NO. 3, are examples of variants in accordance with the present invention. Additionally, NS2 deletion mutants comprising any number of amino acid deletions between the native sequence of NS2 (810-1026) and truncated NS2 (907-1026) are also contemplated to be variants in accordance with the present invention.

### NS3 Product

As used herein, the term “NS3 protease product” refers to NS3 protease domain that is cleaved or released from the NS2/3 protease. NS3 product may correspond with native NS3 (1027-1615), the NS3 protease domain (1027-1206), or may be a functionally equivalent variant thereof, i.e., a variant that retains NS3 protease activity. NS3 product may also correspond to a non-functional variant devoid of NS3 protease activity (such as a S1165A mutant). In one embodiment in a construct described herein, NS3 protease domain is represented by amino acids 1027-1206 [218-397 of SEQ ID NO.1]; however, one of skill in the art will appreciate that NS3 protease product in accordance with the present invention may be modified by insertion, deletion, modification, substitution of one or more amino acids as described above. It is anticipated that such modifications will correspond with modifications existing in the NS3 domain of the NS2/3 protease utilized in the assay. The term “NS3 protease product” is interchangeably used herein with the terms “NS3 product”, “NS3 protease,” or “cleaved NS3 product.”

Accordingly, NS3 truncations comprising amino acid deletions, termed “NS3 fragment” such as fragments: 1027-1187 up to 1027-1205, are examples of variants in accordance with the present invention [NS3 fragment long enough to allow NS2/3 autocleavage but NS3 protease activity is not required]. Additionally, NS3 deletion mutants comprising any number of amino acid deletions between the native sequence of NS3 (1027-1206) and each truncation of NS3 from 1027-1187 up to 1027-1205) (when leading to an active NS2/3 protease) are also contemplated to be variants in accordance with the present invention.

Other useful proteins, enzymes or products of this invention are those linked to an affinity tag to facilitate isolation/separation in the reaction vessel without having to resort to physical separation and transfer to another vessel.

### Affinity Tag

The term “affinity label” or “affinity tag”, as used herein, means a ligand whose affinity for a receptor (or a complementary ligand) can be used to extract (e.g., from a solution) or specifically trap the entity to which the ligand is covalently attached. Affinity tags are indispensable tools that were developed to facilitate the detection and purification of recombinant proteins. They can be classified in two categories: 1) affinity tags that use peptide or protein fusions which bind to small molecule ligands linked to a solid support (hexahistidine tag binding to immobilized transition metals such as nickel, or GST binding to glutathione); or 2) peptide tags binding to an immobilized protein-binding partner (including antibodies) such as the FLAG-tag, the calmodulin-binding peptide, the Strep-tag or Strep-tag II and the biotin acceptor peptide. Examples of pairs of affinity tag/affinity ligand include but are not limited to: Maltose-Binding Protein (MBP)/maltose; Glutathione S Transferase (GST)/glutathione; histidine (His)/metal; avidin/biotin; Strep tag/streptavidin or neutravidin. The metal used as affinity ligand may be selected from the group consisting of: cobalt, zinc, copper, iron, and nickel. The affinity label may be positioned on the N- or C-terminal end of the protein, or in the middle, but particularly on the N-terminus of the protein. Particularly, the metal selected is nickel. The affinity ligand can be set up in columns to facilitate separation by affinity chromatography. For reference, a review paper was recently published (6).

### Specific Ligand

The terms “-specific ligand,” “-selective ligand,” “-directed ligand,” or “-preferential ligand” as used herein mean any molecule that binds to another target molecule with specificity. In the context of the present invention a ligand that would bind to the NS2 or NS3 product can be an antibody that has been raised against a specific portion (peptide) of the NS2 or NS3 proteins which has minimal cross-reactivity with other molecules present in the same reaction vessel, i.e., the uncleaved NS2/3 protease and the other cleavage product.

The term “antibody” as used herein means an immunoglobulin molecule that has a specific amino acid sequence by virtue of which it interacts selectively with the antigen that induced its synthesis in cells of the lymphoid system or with antigens closely related to it. Such antibodies can be polyclonal or monoclonal, the latter of which is made from a single producing clone.

In particular, the terms “-specific antibody,” “-selective antibody,” “-directed antibody,” or “-preferential antibody” as used herein interchangeably mean that the antibody would yield a signal that is at least about 2 fold higher (i.e., signal window) with its target than with the other cleaved product and the uncleaved NS2/3. More particularly, the selective antibody has a signal window that is about 5 fold or higher. Most particularly, the selective antibody has a signal window that is about 15 fold or higher.

Other useful ligands of this invention are those linked to a detectable label to facilitate detection and measurement.

### Detectable Label

As used herein, the terms “label,” “detectable label,” or “detectable marker” refer to any group that may be linked to the specific ligand to allow recognition either directly or indirectly of the resulting ligand-bound molecule such that it can be detected, measured and quantified. Examples of such “labels” include, but are not limited to, fluorescent labels, chemiluminescent labels, calorimetric labels, enzymatic markers, radioactive isotopes, and affinity tags, such as biotin. Such labels are attached to the peptide or antibody by well known methods. A label, or multiple labels, of the present invention can be introduced at any position on the peptide, for example, the label can be at either the C- or N-terminus or within the peptide or antibody, so long as it does not disturb its functional property of recognizing its specific target molecule.

Practical and useful detectable labels are radioactive labels such as ¹²⁵I, fluorescent labels, such as fluorescein or lanthanide-complex (i.e., Eu⁺³), or calorimetric labels, such as horseradish peroxidase or β-galactosidase, and their respective substrate. Such other detectable labels may be found in the Invitrogen-Molecular Probes Handbook—A Guide to Fluorescent Probes and Labeling Technology, 10^{th} ed. 2005 or A guide to HTS Assay Development, D&MD publications ed. April 2004 (and references therein).

As used herein, the term “detergent” means an amphipathic, surface active molecule with polar and non-polar domains. They bind strongly to hydrophobic molecules or molecular domains to confer water solubility. Examples of detergents include, but are not limited to: sodium dodecyl sulphate (SDS), fatty acid salts, the Triton® family, octyl glycoside, 3-[(3-cholamidopropyl)dimethyl-ammonio]-1-propanesulfonate (CHAPS), sodium dodecyl maltoside (DM), lauryldiethylamine oxide (LDAO), NP-40 and the Tween® family.

As used herein, the term “inhibit” or “inhibitor,” when used in reference to the NS2/3 protease, is intended to mean that the protease's ability to autocleave is decreased. Drugs or ligands that can inhibit NS2/3 protease (hereinafter referred to as potential “inhibitors”) may be useful for modulating HCV infection in a population of cells and, therefore, may be useful as medicaments for treating a pathology characterized by the presence of HCV in the cells.

### Preferred Embodiments

In a particular embodiment of the present invention, there is also provided a method for detecting NS3 product in a sample containing NS2/3 protease, comprising:
a) subjecting the sample to suitable conditions under which at least a portion of the NS2/3 protease is cleaved to yield cleaved NS2 and NS3 products;
a′) diluting the protease in said sample to achieve conditions suitable to stop auto-cleavage;
a″) immobilizing the NS3 product from step a′);
b) incubating the immobilized NS3 product of step a″) with a ligand directly or indirectly labeled with a detectable marker wherein said ligand preferentially binds to the NS3 over NS2/3;
c) detecting the immobilized NS3-bound ligand produced in step b), whereby the amount of ligand detected correlates with the NS2/3 autocleavage activity.

In a further embodiment of the present invention, there is also provided a method of detecting NS2 product in a sample containing NS2/3 protease, comprising:
a) subjecting the sample to suitable conditions under which at least a portion of the NS2/3 protease is cleaved to yield cleaved NS2 and NS3 products;
a′) diluting the protease in said sample to achieve conditions suitable to stop auto-cleavage;
a″) immobilizing the NS2 product from step a′);
b) incubating the immobilized NS2 product of step a″) with a ligand directly or indirectly labeled with a detectable marker, wherein said ligand preferentially binds to the NS2 over NS2/3;
c) detecting the NS2-bound ligand produced in step b), whereby the amount of ligand detected correlates with the NS2/3 autocleavage activity.

The methods of the present invention are useful as screening assays to identify candidate drug compounds that have NS2/3 inhibitory activity. Thus, the assays of the present invention may be conducted in the presence or absence of a candidate compound to determine if the candidate compound affects NS2/3 autocleavage. A decrease of detectable NS2 product or NS3 product in the presence of a candidate compound is indicative of NS2/3 inhibition.

Similarly, in another aspect of the present invention there is provided an assay for screening a candidate compound for NS2/3 cleavage inhibitory activity in a sample containing NS2/3 protease, the assay comprising:
a) subjecting a first sample comprising NS2/3 protease to suitable conditions under which at least a portion of NS2/3 is cleaved to yield cleaved NS2 and NS3 products, in the absence of candidate compound;
a′) diluting the protease in said first sample to achieve conditions suitable to stop auto-cleavage;
a″) immobilizing the NS3 product from step a′);
b) subjecting a second sample comprising NS2/3 protease in the presence of a candidate compound under the same conditions as those in step a);
b′) diluting the protease in said second sample to achieve conditions suitable to stop auto-cleavage;
b″) immobilizing the NS3 product, if any, from step b′);
c) incubating immobilized NS3 from step a″) with a ligand directly or indirectly labeled with a detectable marker, wherein said ligand preferentially binds to NS3 over NS2/3;
c′) incubating immobilized NS3 from step b″) with a ligand directly or indirectly labeled with a detectable marker, wherein said ligand binds preferentially to NS3 over NS2/3;
d) determining the amount of immobilized labeled-ligand produced in each of step c) and c′), whereby a decrease in the amount of immobilized labeled-ligand in the step c′) as compared to that for step c) indicates that the candidate compound may be an inhibitor of NS2/3 cleavage activity.

Similarly, in another aspect of the present invention there is provided an assay for screening a candidate compound for NS2/3 cleavage inhibitory activity in a sample containing NS2/3 protease, the assay comprising:
a) subjecting a first sample comprising NS2/3 protease to suitable conditions under which at least a portion of NS2/3 is cleaved to yield cleaved NS2 and NS3 products, in the absence of candidate compound;
a′) diluting the protease in said first sample to achieve conditions suitable to stop auto-cleavage;
a″) immobilizing the NS2 product from step a′);
b) subjecting a second sample comprising NS2/3 protease in the presence of a candidate compound under the same conditions as those in step a);
b′) diluting the protease in said second sample to achieve conditions suitable to stop auto-cleavage;
b″) immobilizing the NS2 product, if any, from step b″);
c) incubating immobilized NS2 from step a″) with a ligand directly or indirectly labeled with a detectable marker, wherein said ligand preferentially binds to NS2 over NS2/3;
c′) incubating immobilized NS2 from step b″) with a ligand directly or indirectly labeled with a detectable marker, wherein said ligand preferentially binds to NS2 over NS2/3;
d) determining the amount of immobilized labeled-ligand produced in each of step c) and c′), whereby a decrease in the amount of immobilized labeled-ligand in the step c′) as compared to that for step c) indicates that the candidate compound may be an inhibitor of NS2/3 cleavage activity.

### NS2/3 Protease and Variants

The NS2/3 protease 810-1206 [SEQ ID NO.1] as well as functionally equivalent variants can be used in embodiments of the present invention. Particularly, examples of variants in accordance with the present invention, are: (907-1206; SEQ ID NO. 2) or (904-1206; SEQ ID NO. 3). Particularly, NS2/3 variant (4K-6H-904-1206-ST-4K) of SEQ ID NO. 4 is used in the assay of the present invention.

### NS2/3 Autocleavage Assay Conditions

In a first step of the present method, a sample is subjected to conditions under which NS2/3 is cleaved to yield a NS2 product and a NS3 product. Such conditions, including the use of a detergent as an activation agent, are known in the art (2-5 all incorporated herein by reference) and suitable conditions are also exemplified herein.

Activation of the refolded NS2/3 protease requires the use of detergents at concentrations at or above their critical micelle concentration, although some detergents do not promote autocleavage. Also, the effect of the detergent on NS2/3 autocleavage activity is enhanced in the presence of glycerol (2). The concentration dependence of the NS2/3 protease autocleavage reaction previously reported (4) is confirmed using SDS-PAGE/Western blot analysis. At concentrations greater than 200 nM, no concentration dependence is observed (data not shown). The effect of glycerol, pH and DMSO on autocleavage is also evaluated. Similar cleavage kinetics is observed in a buffer containing 20% or 30% glycerol (data not shown). Finally, autocleavage is optimal at pH 7.5 and DMSO has no effect on activity at concentrations ranging from 0.5-5% (data not shown).

Particularly, the NS2/3 is originally prepared in a solution of LDAO to prevent self-cleavage prior to the start of the assay. Particularly, the concentration of LDAO should be well above critical micelle concentration (CMC) in order to block autocleavage. More particularly, in the present assay conditions, LDAO should be present between 0.5 and 1.5% in the solution, most particularly, at about 1%. The NS2/3 solution is afterwards diluted in a solution lacking LDAO, to achieve lower concentrations in order for autocleavage to proceed.

The autocleavage reaction is therefore induced by decreasing the concentration of LDAO, in the presence of an activation agent, the activation agent being a detergent selected from, but not limited to, the group consisting of: CHAPS, Triton X-100, NP-40 and n-dodecyl- -D-maltoside (DM). Typically, the detergent acting as activation agent is present above its CMC.

Typically, glycerol is present to enhance autocleavage, particularly from 0% to 50%, more particularly, from 20% to 50%, most particularly at 20%.

The NS2/3 protease auto-cleavage is then stopped by transferring an aliquot of the reaction sample in a second reaction vessel thereby diluting the autocleavage reaction mixture and stopping autocleavage. The transfer contributes to stop autocleavage by 1) decreasing the NS2/3 protease concentration, and 2) diluting the amount of activation agent.

In one particular embodiment, the reaction is stopped with a 5-fold dilution of the autocleavage reaction mixture in a buffer containing 50 mM HEPES, pH 7.5, 10% glycerol and 1 mM TCEP.

### Immobilization

Upon transfer of the cleavage mixture containing NS2 product, NS3 product and any remaining uncleaved NS2/3, the cleavage mixture while being diluted (and auto-cleavage thereby stopped) is also preferably immobilized for the purpose of detection using standard means of immobilization. In one embodiment, the NS2/3 protease precursor is tagged on the NS2 portion so as to facilitate immobilization of the cleaved NS2 product which retains the tag on cleavage (any remaining uncleaved NS2/3 will also be immobilized). In another embodiment, the NS2/3 protease precursor is tagged on the NS3 portion. In this case, the cleavage reaction mixture is exposed to an immobilizing surface to which the tagged NS3 will readily bind. Particularly, the corresponding affinity receptor may be coated on the reaction vessel or immobilizing surface to facilitate multiple washings without disrupting the labeled product to be measured.

Preferable pairs of affinity tag/affinity receptor are selected from: MBP/maltose; GST/glutathione; His/Ni; Strep tag/streptavidin, or neutravidin.

Particularly, the NS2 domain is tagged on the N-terminus with a hexahistidine tag and the reaction vessel is coated with nickel. Preferably, the NS3 domain is tagged on the C-terminus with a Strep tag and the reaction vessel is coated with streptavidin or neutravidin. Particularly, the reaction takes place in 96 well or 384 well plates which have been previously coated with the appropriate affinity receptor. Alternatively, these plates can be purchased from a commercial source (i.e., Pierce).

### Specific Ligand

Once the cleavage mixture (either of NS2 or NS3 cleavage product and any remaining uncleaved NS2/3) is immobilized, it is combined with a suitable amount of a specific ligand that preferentially recognizes cleaved NS2 or NS3 over NS2/3 under conditions suitable to permit preferential binding of the specific ligand. Preferential binding of the ligand to the NS2 product or NS3 product and low binding to the uncleaved NS2/3 protease is preferable for an accurate determination of cleavage.

Particularly, the specific ligand is directed against the NS2 cleavage product. Alternatively, the ligand is directed against the NS3 cleavage product. As would be known in the art, the amount of NS2- or NS3-selective ligand to be used in the assay may be determined based on the total possible amount of cleaved NS2 or NS3 that may result in the reaction.

In a particular aspect, the specific ligand is an antibody. Particularly, the antibody is a polyclonal antibody or a monoclonal antibody. Examples of NS2 preferential ligands include antibodies directed to specific amino acid sequences of NS2. Particularly, the specific amino acid sequence used to raise antibodies has a length that is sufficient to induce an immune response and a sequence that is appropriate to raise antibodies that are selective against the NS2 product, i.e., that will have low or no cross-reactivity with the NS3 product and the uncleaved NS2/3. Of course, as will be recognized by a person of skill in the art, such short peptides may need to be conjugated to a carrier protein in order to induce an immune response (as is presented in Example 2 hereinbelow).

In particular, these peptides are selected from the NS2 portion of the protein and may be found by assessing multiple straddling peptides comprising at least 10 consecutive amino acids. Without intending to be limited, for example, short peptides taken from SEQ ID NO.1 may be conjugated and injected to induce an immune response selective for NS2.

In particular, the peptides comprising the following amino acid sequences may be used in accordance with the invention:

[SEQ ID NO. 6]

Ser-Phe-Glu-Gly-Gln-Gly-Trp-Arg-Leu-Leu

[SEQ ID NO. 8]

Asn-Phe-Glu-Gly-Gln-Gly-Trp-Arg-Leu-Leu;

[SEQ ID NO. 9]

Asp-Asn-Phe-Glu-Gly-Gln-Gly-Trp-Arg-Leu;

[SEQ ID NO. 10]

Ala-Asp-Asn-Phe-Glu-Gly-Gln-Gly-Trp-Arg;

[SEQ ID NO. 11]

Pro-Ala-Asp-Asn-Phe-Glu-Gly-Gln-Gly-Trp;

[SEQ ID NO. 12]

Gly-Pro-Ala-Asp-Asn-Phe-Glu-Gly-Gln-Gly;

[SEQ ID NO. 13]

Ser-Ala-Arg-Arg-Gly-Arg-Glu-Ile-Leu-Leu;
etc.

Other peptides useful to raise antibodies according to the invention will be readily determined by persons of skill in the art.

In an alternative aspect, the preferential ligand is an antibody directed towards a specific amino acid sequence of NS3. Particularly, the antibody is a polyclonal antibody or a monoclonal antibody. Examples of NS3 preferential ligands include antibodies directed to specific amino acid sequences of NS3. Particularly, the specific amino acid sequence used to raise antibodies has a length that is sufficient to induce an immune response and a sequence that is appropriate to raise antibodies that are selective against the NS3 product, i.e., that will have low or no cross-reactivity with the NS2 product and the uncleaved NS2/3. Of course, as will be recognized by a person of skill in the art, such short peptides may need to be conjugated to a carrier protein in order to induce an immune response (as is presented in Example 2 hereinbelow).

In particular, these peptides are selected from the NS3 portion of the protein and may be found by assessing multiple straddling peptides comprising at least 10 consecutive amino acids. Without intending to be limited, for example, short peptides from SEQ ID NO.1 may be conjugated and injected to induce an immune response selective for NS3.

In particular, the peptides comprising the following amino acid sequences may be used in accordance with the invention:

[SEQ ID NO. 5]

Ala-Pro-Ile-Thr-Ala-Tyr-Ser-Gln-Gln-Thr;

[SEQ ID NO. 14]

Pro-Ile-Thr-Ala-Tyr-Ser-Gln-Gln-Thr-Thr;

[SEQ ID NO. 15]

Ile-Thr-Ala-Tyr-Ser-Gln-Gln-Thr-Thr-Arg;

[SEQ ID NO. 16]

Thr-Ala-Tyr-Ser-Gln-Gln-Thr-Thr-Arg-Gly;

[SEQ ID NO. 17]

Arg-Gly-Leu-Leu-Gly-Cys-Ile-Ile-Thr-Ser;
etc.

Other peptides useful to raise antibodies according to the invention will be readily determined by persons of skill in the art.

In one embodiment, the antibody directed against an N-terminal region of NS3 is used as the NS3 preferential ligand, for example, an antibody which is directed against the peptide, APITAYSQQT [SEQ ID NO.5], particularly, the K147 polyclonal antibody.

In particular embodiments, the preferential ligand is either a polyclonal antibody or a monoclonal antibody. Particularly, the polyclonal or monoclonal antibody is coupled to a detectable label to facilitate detection of its target molecule. Particularly, the ligand can be detected directly, if it is directly coupled to a detectable label, or alternatively, the specific antibody can be detected indirectly with the use of a second antibody directed against the specific antibody, this second antibody being coupled to a detectable label. Such second antibody can be polyclonal or monoclonal antibodies and can be selected from: monoclonal antibodies (such as anti-IgG, anti-IgM) or polyclonal antibodies (such as: anti-rabbit, anti-mouse, or anti-goat antibodies, etc.) depending on the nature of the specific ligand used to detect the cleavage product. In a particular embodiment, the specific ligand is a polyclonal antibody from rabbit which is detected with the use of an anti-rabbit antibody labeled with Europium.

In another preferred embodiment, the amount of immobilized ligand produced from each of steps c) and c′) can be determined by measuring the signal of the label bound directly or indirectly to the immobilized ligand.

### Detection

In a particular embodiment of this invention, the NS2- or NS3-preferential ligand is linked (directly or indirectly) to any conventionally used detectable label in order that cleaved NS2 product or NS3 product may be identified and/or quantified. It follows that conventional methods of detection can then be used to detect/measure the detectable label that is bound to immobilized NS2 or NS3. Embodiments of such detectable labels include, for example, radioactive or calorimetric labels that are well known in the art and available in catalogs such as “Amersham Blotting, Labeling and Detection” catalog, GE Healthcare at www.Amershambiosciences.com.

In one embodiment, the detectable label is a fluorescent marker. The detection/measurement of this detectable label is carried out by methods well known in the art such as is disclosed in “Invitrogen-Molecular Probes Handbook—A Guide to Fluorescent Probes and Labeling Technology, 10^{th} ed. 2005.”

Alternatively, the detectable label is europium that is bound to an anti-rabbit antibody that recognizes the anti-NS3 rabbit antibody referred to above. The detection/measurement of this detectable label is carried out by methods well known in the art such as commercialized by PerkinElmer Life Sciences (DELFIA® system).

It is to be understood in the present embodiment and in the various other embodiments disclosed and claimed herein that the general conditions, including buffers employed, pH of buffers and solutions employed, temperatures employed, and time of reaction would include those that do not inhibit the intended various steps and would be readily determinable by persons skilled in the art.

Embodiments of the invention are described by reference to the following specific examples which are not to be construed as limiting:

### EXAMPLES

### Abbreviations

Ab: antibody;
BSA: Bovine serum albumin;
CHAPS: 3-[(3-chloroamidopropyl)dimethyl-ammonio]-1-propanesulfonate;
DMSO: dimethyl sulfoxide;
DM: n-dodecyl-β-D-maltoside;
DTPA: diethylenetriaminepentaacetic acid;
HEPES: 4-(2-hydroxyethyl)-1-piperazineethanesulfonic acid;
LDAO: lauryldiethylamine oxide;
mcKLH: Mari culture keyhole limpet hemocyanin;
NZW: New Zealand White rabbit;
PBS: phosphate buffered saline;
PBS-T: phosphate buffered saline-Tween;
PNPP: para-nitrophenylphosphate;
PVDF: polyvinylidene difluoride;
TCEP: Tris(2-carboxyethyl)phosphine hydrochloride.

### Materials and Methods

### Assay Reagents

BSA, glycerol, DTPA, zinc chloride, HEPES and DMSO were purchased from Sigma-Aldrich. The detergents DM and LDAO were from Anathrace Inc. and Fluka respectively. DELFIA® reagents were purchased from PerkinElmer Life Sciences. TCEP was from Pierce, Tween®20 from Bio-Rad and sodium chloride from EM Science.

### NS2/3 Protease Preparation

The expression, production and purification of the NS2/3 protease was carried out according to the procedure previously reported (2). Practically speaking, aliquots of the refolded, inactive NS2/3 protein in refolding buffer (50 mM Tris, pH 8.0, 0.5 arginine HCl, 1% LDAO, 5 mM TCEP) can be stored frozen at −80° C., and later thawed and diluted in the presence of an activation agent and optionally glycerol, to induce autocleavage.

### Generation of Polyclonal Antibodies

An important aspect of this invention is the use of antibodies as ligand to preferentially bind NS2 or NS3 over NS2/3 in the presence of a mixture of the two.

In order to obtain an antibody that recognizes cleaved NS2 from NS2/3, a peptide corresponding to the C-terminal last 10 amino acid sequence of NS2 is synthesized. The synthetic peptide (SFEGQGWRLL; SEQ ID NO.6) is coupled to a carrier protein and used for immunization.

In order to obtain an antibody that recognizes cleaved NS3 from NS2/3, a peptide corresponding to the N-terminal first 10 amino acid sequence of NS3 is synthesized. The synthetic peptide (APITAYSQQT) [SEQ ID NO.5] is coupled to a carrier protein and used for immunization.

### Example 1

### Generation of NS3-Specific Polyclonal Antibodies (K147)

In order to obtain a polyclonal antibody that recognizes cleaved NS3 from NS2/3, a peptide corresponding to the N-terminal first 10 amino acid sequence of NS3 (APITAYSQQT; SEQ ID NO.5) is coupled to keyhole limpet hemocyanin (mcKLH) carrier protein and used to immunize rabbits.

### Peptide Synthesis and Immunogen Preparation

The peptide H2N-APITAYSQQT-COOH is purchased from Neo MPS, Inc. (San Diego, Calif.). To prepare the immunogen, the peptide is conjugated to Mari culture keyhole limpet hemocyanin (mcKLH) carrier protein using the Imject® Immunogen EDC conjugate kit from Pierce. Essentially, 2 mg of the peptide are solubilized in 0.5 ml of Imject® EDC conjugation buffer. The peptide solution is added to 0.2 ml of the reconstituted mcKLH carrier protein solution. Fifty (50) μl of freshly prepared EDC reagent at 10 mg/ml is added to the conjugation reaction and the reaction is then incubated for 2 hours at room temperature. The conjugate is purified by desalting using the desalting column and purification buffer provided in the Imject® immunogen EDC Kit. The fractions containing the immunogen (determined by OD280) are pooled and stored at −20° C.

### Immunization

The peptide-carrier protein conjugate is diluted in PBS to achieve 50 μg/ml and emulsified with an equal volume of complete Freund's adjuvant. Two NZW (New Zealand White) male rabbits are immunized sub-cutaneously (s.c.) with the emulsion (50 μg/rabbit in a volume of 2 ml). The rabbits are boosted with the same dose of peptide conjugate emulsified in incomplete Freund's adjuvant at week 4 and 8. Blood is collected 10-14 days after each booster injection and tested for peptide specific antibodies by ELISA. Pre-immune serum is collected one day before the first immunization to use as control serum for each animal. The sera from each rabbit is analyzed by Western blot and by ELISA using peptide antigen-coated plates.

### ELISA Assay

For this assay, microtiter plates (NUNC) are coated with the peptide antigen (100 μl per well of a 12.5 μg/ml solution) overnight at 4° C. The plates are washed three times with 200 μl blocking buffer containing PBS, 3% BSA and 0.05% Tween-20 and incubated for one hour with 200 μl of blocking buffer at room temperature. The wells are washed three times with PBS containing 0.05% Tween-20 (PBS-T). The wells containing the peptide are then incubated with serial dilutions of the rabbit antiserum (1/5-1/390,625) in PBS-T for 2 hours at room temperature. A dilution curve for the pre-immune serum is also tested in parallel. The wells are washed 3 times with 200 μl PBS-T and then incubated with 100 μl of goat anti-rabbit alkaline phosphatase conjugate (1/5000) (Gibco BRL) for 1 hour at room temperature. The wells are washed twice with PBS-T and rinsed with 200 μl of PNPP buffer. This step is followed by an incubation with 100 μl of 4-nitrophenyl phosphate (5 ng/ml diluted in PNPP buffer). The optical absorbance is read at 405 nm.

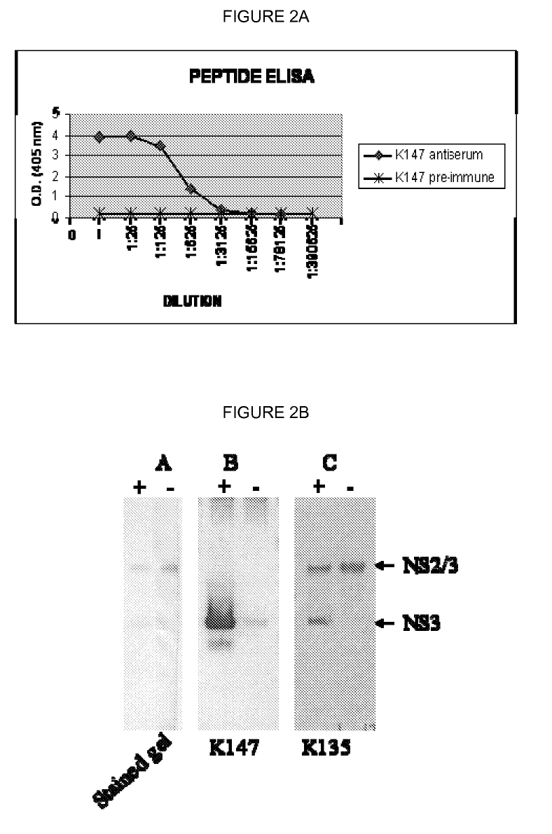

FIG. 2A illustrates the antibody dilution curve obtained from the bleed from one animal following the second boost and shows that the antiserum reacts with the peptide antigen in the ELISA. The titer of the antisera against the peptide (1:625) antigen is maintained between the first and second boost. Both immunized rabbits raise an immune response against the peptide antigen as measured by ELISA.

### Western Blot

NS2/3 protein samples are submitted to the autocleavage reaction and used to evaluate the ability of the polyclonal antibody to recognize preferentially NS3 cleaved product from the NS2/3 protease by Western blot. Protein samples (100 ng) before (−) and after (+) NS2/3 autocleavage reaction are separated by electrophoreses on 15% polyacrilamide gels. The proteins are transferred onto a PVDF membrane by electroblotting. The membranes are blocked with 5% skim milk in PBS-Tween (phosphate buffer saline with 0.05% Tween-20). The membrane is then incubated with the primary antibody for 1 hour at room temperature. The K147 antiserum is diluted 1:1000 while the control polyclonal antibody to NS3 [K135] (Thibeault et al., 2001) is diluted at 1:5000. After washing 4 times, the blots are incubated with 1:20,000 goat anti-rabbit HRP-conjugated secondary antibody (Gibco BRL) for 1 hour at room temperature. After 4 washes, the reaction is visualized using ECLplus western blotting substrate from Pierce and the chemiluminescence signal read on a STORM® Image analysis system (Amersham).

The signal observed in FIG. 2B with the samples incubated with the K147 antiserum to the N-terminus of NS3 demonstrates that this antibody can recognize preferentially NS3 when cleaved (+) from NS2/3 protease since no significant signal is observed in lane (−) where the uncleaved NS2/3 is recognized by the control anti-NS3 antibody directed against the catalytic domain (K135; panel C). The selectivity of this antiserum is further demonstrated in the 96 well format for NS2/3 autocleavage assay described in Example 2.

From the two rabbits that produce an immune response, one rabbit produces an antiserum that is selective for NS3.

The immunization protocol is then repeated a second time with 6 rabbits. This time, 4 rabbits produce an antiserum that is selective for NS3.

### Example 2

### Evaluation of Polyclonal Antibody (K147)

The assay conditions described below are used to evaluate polyclonal antibodies for their ability to discriminate between NS2/3 protease and the NS3 product.

### NS2/3 Protease Autocleavage

The autocleavage reaction is initiated by adding 20 μL of autocleavage buffer (50 mM HEPES, pH 7.5, 30% glycerol, 0.5% DM, 1 mM TCEP) to 30 μL of NS2/3 protease (SEQ ID NO.4 diluted to a final concentration of 0.2 μM in 50 mM HEPES, pH 7.5, 30% glycerol, 1 mM TCEP). The reaction mixture is incubated for 90 minutes at 30° C. In the blank reaction, the DM-containing buffer is added just prior to the transfer step. Alternatively, as a negative control, the active-site mutated NS2/3 [H952A] is used to confirm that the antibody minimally cross-reacts with uncleaved NS2/3.

Antibody Evaluation for the Detection Step with a Eu+3-Labeled Anti-Rabbit Ab

In a 96-well neutravidin coated plate (purchased from Pierce), 20 μL of the autocleavage mixture is added to 80 μL of 50 mM HEPES, pH 7.5, 10% glycerol, 1 mM TCEP. The assay mixture is incubated for 60 min at room temperature. The plate is then washed three times with 200 μL PBS, 0.05% Tween-20. Then, 100 μL PBS, 0.05% Tween-20, 3% BSA is added per well followed by a 30-min incubation at room temperature and three washes as described above.

100 μL of the polyclonal antibody diluted 1/500 in PBS, pH 7.5, 0.05% Tween-20, 0.3% BSA is added per well followed by a 30-min incubation at room temperature. The plate is then washed three times as described above.

To detect binding of the polyclonal antibody, 100 μL of DELFIA® Eu-N1 labeled anti-rabbit antibody (PerkinElmer Life Sciences) diluted to 8 nM in PBS, pH 7.5, 0.05% Tween-20, 0.3% BSA, 100 μM DTPA is added per well followed by a 30-min incubation at room temperature. The plate is then washed three times with 200 μL of the DELFIA® Wash Buffer (PerkinElmer Life Sciences). Finally, 100 μL of the DELFIA® Enhancement Solution (PerkinElmer Life Sciences) is added to each well followed by an incubation of at least 15 min at room temperature. The time-resolved fluorescence is monitored on a Wallac Victor® 1420 Multilabel HTS Counter (PerkinElmer Life Sciences) equipped with an excitation filter at 340 nm and an emission filter at 615 nm.

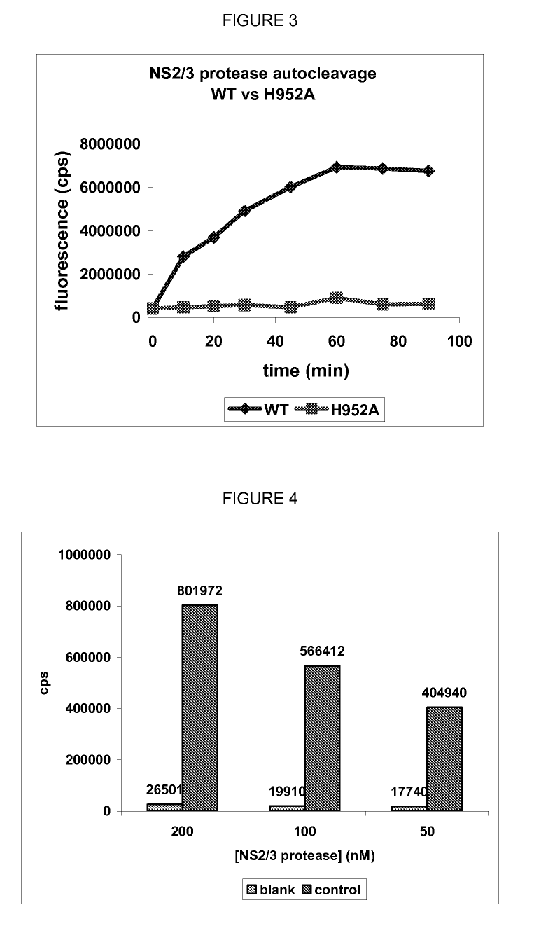

Using this assay format, an increase in fluorescence is observed upon NS2/3 autocleavage, while no increase in fluorescence is observed with the NS2/3 protease active-site mutant H952A (FIG. 3) indicating that K147 is selective for cleaved NS3 and that its detection correlates with NS2/3 autocleavage activity.

### Example 3

### Protocol for NS2/3 Protease Time-Resolved Fluorescence Assay

The autocleavage reaction is initiated by adding 10 μL of NS2/3 protease (SEQ ID NO.4 diluted to a final concentration of 800 nM in 50 mM HEPES, pH 7.5, 20% glycerol, 1 mM TCEP) to 30 μL of 50 mM HEPES, pH 7.5, 20% glycerol, 0.266% n-dodecyl-β-D-maltoside, 1 mM TCEP with the final DMSO content kept at 5%. The reaction mixture is incubated for 45 minutes at room temperature.

In the blank wells, autocleavage is prevented by adding ZnCl₂ at 10 μM or NS4A peptide [SEQ ID NO. 7] at 50 μM.

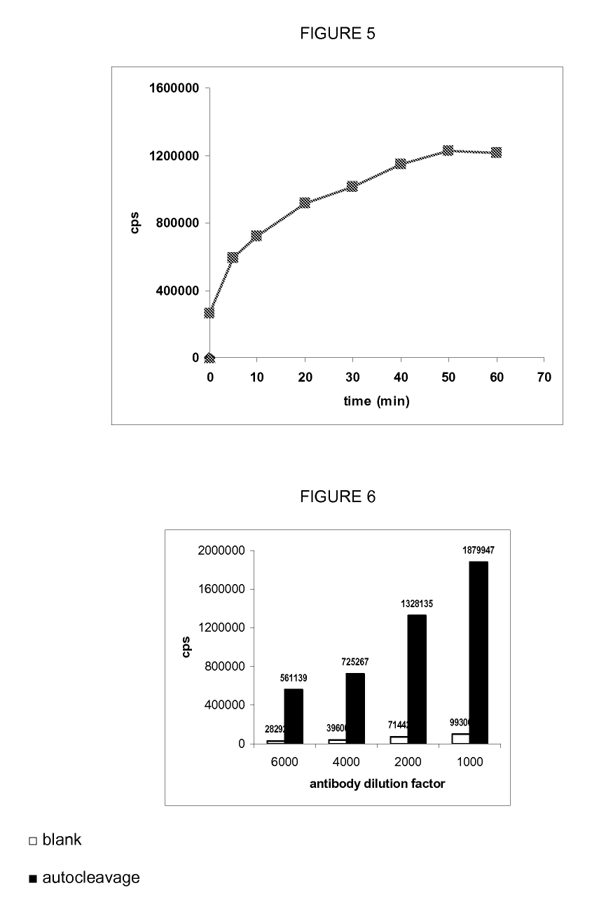

A final NS2/3 protease concentration of 200 nM is selected based on the concentration dependence of the autocleavage as shown in FIG. 4. A time-course of the autocleavage reaction is shown in FIG. 5.

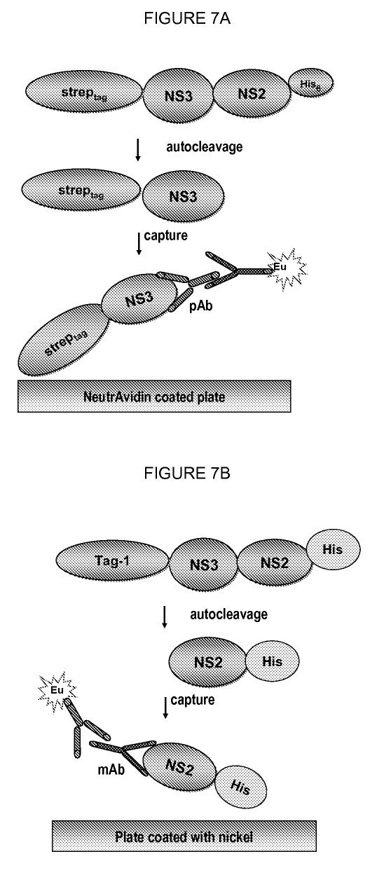

In a 96-well neutravidin-coated plate (purchased from Pierce), 10 μL of the autocleavage mixture is added to 40 μL of 50 mM HEPES, pH 7.5, 10% glycerol, 1 mM TCEP. The assay mixture is incubated for 60 min at room temperature. The plate is then washed three times with 100 μL of 50 mM HEPES, pH 7.5, 0.15 M NaCl, 0.05% Tween-20. Then, 50 μL of the polyclonal antibody K147 diluted up to 6000-fold in 50 mM HEPES, pH 7.5, 0.15 M NaCl, 0.05% Tween®-20, 0.3% BSA is added per well followed by a 30-min incubation at room temperature. The plate is then washed three times as described above. Then, 50 μL of 1.0 nM DELFIA Eu-N1 labeled anti-rabbit antibody (PerkinElmer Life Sciences) diluted in 50 mM HEPES, pH 7.5, 0.15 M NaCl, 0.05% Tween®-20, 0.3% BSA, 100 μM DTPA is added per well followed by a 30-min incubation at room temperature. The plate is then washed three times with 100 μL of the DELFIA® wash buffer. Finally, 50 μL of the DELFIA® enhancement solution is added to each well followed by an incubation of at least 15 min at room temperature. The time-resolved fluorescence is monitored on a Wallac Victor® 1420 Multilabel HTS Counter (PerkinElmer Life Sciences) equipped with an excitation filter at 340 nm and an emission filter at 615 nm. An increase in fluorescence is observed with increasing concentrations of the antibody K147 and with control-to-blank ratios ranging from 18 to 20, clearly showing the ability of the antibody to discriminate between the NS2/3 precursor and the NS3 product (FIG. 6). A schematic representation of the assay is shown in FIG. 7A.

In conclusion, in the NS2/3 protease time-resolved fluorescence assay presented herein, the NS2/3 uncleaved precursor and the NS3 product are both captured on the neutravidin-coated plate via their C-terminal Strep-tag, and the NS3 product is detected by using a rabbit polyclonal antibody able to discriminate between the NS2/3 precursor and the NS3 product and an europium-labeled anti-rabbit antibody.

### Example 4

### Immobilization Assay Based on NS2 Detection

Likewise to detect the NS2 product, an antibody is raised against the peptide SFEGQGWRLL (SEQ ID NO. 6).

The autocleavage reaction is initiated by adding in a 96-well round-bottom polypropylene plate (Falcon) 10 μL of NS2/3 protease (SEQ ID NO.4 diluted to a final concentration of 800 nM in 50 mM HEPES, pH 7.5, 20% glycerol, 1 mM TCEP) to 30 μL of 50 mM HEPES, pH 7.5, 20% glycerol, 0.266% DM, 1 mM TCEP with the final DMSO content kept at 5%. The reaction mixture is incubated for 45 minutes at room temperature. In the negative control wells, autocleavage is prevented by adding ZnCl₂ at 10 μM or NS4A peptide (SEQ ID NO. 7) at 50 μM.

In a 96-well nickel-coated plate (purchased from Pierce), 10 μL of the autocleavage mixture is added to 40 μL of 50 mM HEPES, pH 7.5, 10% glycerol, 1 mM TCEP. The assay mixture is incubated for 60 minutes at room temperature. The plate is then washed three times with 100 μL of 50 mM HEPES, pH 7.5, 0.15M NaCl, 0.05% Tween®-20. Then, 50 μL of the anti-NS2 antibody (previously titrated to determine the dilution factor) diluted in 50 mM HEPES, pH 7.5, 0.15M NaCl, 0.05% Tween®-20, 0.3% BSA is added per well followed by a 30-min incubation at room temperature. The plate is then washed three times as described above. Then, 50 μL of 1.0 nM DELFIA® Eu-N1 labeled anti-mouse antibody (PerkinElmer Life Sciences) diluted in 50 mM HEPES, pH 7.5, 0.15M NaCl, 0.05% Tween®-20, 0.3% BSA, 100 μM DTPA is added per well followed by a 30-min incubation at room temperature. The plate is then washed three times with 100 μL of the DELFIA® wash buffer (PerkinElmer Life Sciences). Finally, 50 μL of the DELFIA® enhancement solution (PerkinElmer Life Sciences) is added to each well followed by an incubation of at least 15 minutes at room temperature. The time-resolved fluorescence is monitored on a Wallac Victor® 1420 Multilabel HTS Counter (PerkinElmer Life Sciences) equipped with an excitation filter at 340 nm at an emission filter at 615 nm. A schematic representation of the assay is shown in FIG. 7B.

### Example 5

### Protocol for Ultra High Throughput Screening

The autocleavage reaction is initiated by adding in a 384-well round-bottom polypropylene plate (Greiner): 10 μL of NS2/3 protease (SEQ ID NO.4 diluted to a final concentration of 600 nM in 50 mM HEPES, pH 7.5, 20% glycerol, 1 mM TCEP) to 10 μL of the test compound in DMSO (diluted in 50 mM HEPES, pH 7.5, 20% glycerol, 1 mM TCEP) and 10 μL of 50 mM HEPES, pH 7.5, 20% glycerol, 0.6% DM, 1 mM TCEP. The final DMSO content is kept at 5%. The reaction mixture is incubated for 45 minutes at room temperature. In the blank wells, autocleavage is prevented by adding ZnCl₂ at 10 μM or NS4A peptide (SEQ ID NO. 7) at 50 μM.

In a 384-well neutravidin-coated plate (purchased from Pierce), 5 μL of the autocleavage mixture is added to 20 μL of 50 mM HEPES, pH 7.5, 10% glycerol, 1 mM TCEP. The assay mixture is incubated for 60 minutes at room temperature. The plate is then washed three times with 50 μL of 50 mM HEPES, pH 7.5, 0.15M NaCl, 0.05% Tween®-20. Then, 25 μL of the polyclonal antibody K147 diluted to 0.03 μg./ml in 50 mM HEPES, pH 7.5, 0.15M NaCl, 0.05% Tween®-20, 0.3% BSA is added per well followed by a 30-min incubation at room temperature. The plate is then washed three times as described above. Then, 25 μL of DELFIA® Eu-N1 labeled anti-rabbit antibody (PerkinElmer Life Sciences) diluted to 1.0 nM in 50 mM HEPES, pH 7.5, 0.15M NaCl, 0.05% Tween®-20, 0.3% BSA, 100 μM DTPA is added per well followed by a 30-min incubation at room temperature. The plate is then washed three times with 50 μL of the DELFIA® wash buffer (PerkinElmer Life Sciences).

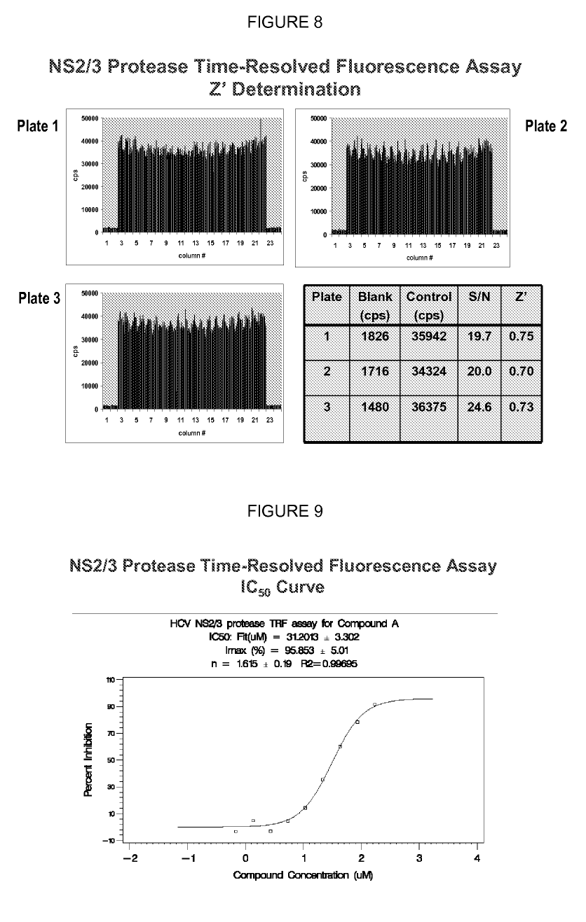

Alternatively, 25 μL of the antibodies solution composed of: 1) 0.03 μg/mL of the K147 polyclonal antibody previously purified on a peptide column using APITAYSQQT as ligand, and 2) 0.5 nM DELFIA® Eu-N1 labeled anti-rabbit antibody (PerkinElmer Life Sciences) diluted in 50 mM HEPES, pH 7.5, 0.15M NaCl, 0.05% Tween®-20, 0.3% BSA, 100 μM DTPA is added per well followed by a 30-min incubation at room temperature. The plate is then washed three times with 50 μL of the DELFIA® wash buffer (PerkinElmer Life Sciences). Finally, 25 μL of the DELFIA® enhancement solution (PerkinElmer Life Sciences) is added to each well followed by an incubation of at least 15 minutes at room temperature. The time-resolved fluorescence is monitored on a Wallac Victor® 1420 Multilabel HTS Counter (PerkinElmer Life Sciences) equipped with an excitation filter at 340 nm at an emission filter at 615 nm. Assay statistics are presented in FIG. 8 where it can be seen that a signal window of 20-25 is observed between the positive control (maximal NS2/3 protease activity) and the blank (background NS2/3 protease activity) with a Z′ of 0.70-0.75 [Z′ being a statistical parameter defined in (7)]. FIG. 9 shows the results obtained with the same assay on test compound A diluted at different concentrations. The level of inhibition (% inhibition) of each well containing inhibitor was calculated with the following equation:

$$\% inhibition = ( 1 - [ \frac{{cps}_{well} - {cps}_{blank}}{{cps}_{control} - {cps}_{blank}} ] ) * 100$$

The percentage of inhibition was plotted against compound A concentration and a nonlinear curve was fitted to the percent inhibition-concentration data according to the Hill model. The calculated percent inhibition values were then used to determine the median effective concentration IC₅₀, the slope factor (n) and the maximum inhibition (Iₘₐₓ) by the NLIN procedure of the SAS software (Statistical Software System; SAS Institute, Inc., Cary, N.C.) using the following equation:

$$\% inhibition = \frac{{I}_{m ax} \times {[ inhibitor ]}^{n}}{{[ inhibitor ]}^{n} + {IC}_{50}^{n}}$$

An IC₅₀ of approximately 31 μM is obtained for compound A.

What is claimed is:

1. A method for detecting a NS2/3 autocleavage product in a sample containing NS2/3 protease, the method comprising:
a) subjecting the sample to suitable conditions under which at least a portion of the NS2/3 protease is self-cleaved to yield cleaved NS2 and NS3 products;
b) incubating the NS2 and NS3 products with a ligand that can preferentially bind to one of either NS2 or NS3 product over NS2/3 under conditions suitable to afford binding of the ligand to NS2 or NS3; and
c) detecting NS2- or NS3-bound ligand produced in step b), whereby the amount of bound ligand detected correlates with the NS2/3 autocleavage activity.

2. The method according to claim 1, wherein LDAO is initially present in said NS2/3 sample to prevent autocleavage.

3. The method according to claim 2, wherein said LDAO is present at a concentration of at least about 1%.

4. The method according to claim 1, wherein in step a) said sample is diluted such that LDAO concentration is decreased at least about 1.5 fold and is incubated in the presence of an activation agent to induce autocleavage.

5. The method according to claim 4, wherein said activation agent is a detergent selected from the group consisting of: CHAPS, Triton X-100, NP-40 and n-dodecyl-β-D-maltoside (DM).

6. The method according to claim 5, wherein said detergent acting as activation agent is present above its CMC.

7. The method according to claim 6, wherein in step a), said protease is incubated in a solution further comprising glycerol to enhance autocleavage.

8. The method according to claim 7, wherein said glycerol is present at a concentration of about 20%.

9. The method according to claim 1, wherein in step b) said ligand is an antibody.

10. The method according to claim 9, wherein said antibody is a polyclonal antibody or a monoclonal antibody.

11. The method according to claim 10, wherein said antibody is an antibody directed to amino acid sequences specific to NS2 cleaved product and said antibody is selective inasmuch as it has no or low cross-reactivity with NS3 cleaved product and uncleaved NS2/3.

12. The method according to claim 11, wherein said NS2-selective antibody is an antibody directed against a peptide comprising an amino acid sequence selected from the groups consisting of:
[SEQ ID NO. 6]
Ser-Phe-Glu-Gly-Gln-Gly-Trp-Arg-Leu-Leu;
[SEQ ID NO. 8]
Asn-Phe-Glu-Gly-Gln-Gly-Trp-Arg-Leu-Leu;
[SEQ ID NO. 9]
Asp-Asn-Phe-Glu-Gly-Gln-Gly-Trp-Arg-Leu;
[SEQ ID NO. 10]
Ala-Asp-Asn-Phe-Glu-Gly-Gln-Gly-Trp-Arg;
[SEQ ID NO. 11]
Pro-Ala-Asp-Asn-Phe-Glu-Gly-Gln-Gly-Trp;
[SEQ ID NO. 12]
Gly-Pro-Ala-Asp-Asn-Phe-Glu-Gly-Gln-Gly;
and
[SEQ ID NO. 13]
Ser-Ala-Arg-Arg-Gly-Arg-Glu-Ile-Leu-Leu.

13. The method according to claim 10, wherein said antibody is an antibody directed to amino acid sequences specific to NS3 cleaved product and said antibody is selective inasmuch as it has no or low cross-reactivity with NS2 cleaved product and uncleaved NS2/3.

14. The method according to claim 13, wherein said NS3-selective antibody is an antibody directed against a peptide comprising an amino acid sequence selected from the group consisting of:
[SEQ ID NO. 5]
Ala-Pro-Ile-Thr-Ala-Tyr-Ser-Gln-Gln-Thr;
[SEQ ID NO. 14]
Pro-Ile-Thr-Ala-Tyr-Ser-Gln-Gln-Thr-Thr;
[SEQ ID NO. 15]
Ile-Thr-Ala-Tyr-Ser-Gln-Gln-Thr-Thr-Arg;
[SEQ ID NO. 16]
Thr-Ala-Tyr-Ser-Gln-Gln-Thr-Thr-Arg-Gly;
and
[SEQ ID NO. 17]
Arg-Gly-Leu-Leu-Gly-Cys-Ile-Ile-Thr-Ser.

15. The method according to claim 14, wherein said antibody is directed against a peptide comprising amino acid sequence: APITAYSQQT [SEQ ID NO.5].

16. The method according to claim 15, wherein said antibody is K147 rabbit polyclonal antibody.

17. The method according to claim 9, wherein said antibody is detected with the use of a detectable label.

18. The method according to claim 17, wherein said detectable label is selected from the group consisting of: fluorescent label, chemiluminescent label, calorimetric label, enzymatic marker, and radioactive isotope.

19. The method according to claim 18, wherein said detectable label is europium.

20. The method according to claim 17, wherein said detectable label is linked directly to said antibody.

21. The method according to claim 17, wherein said detectable label is linked to an antibody that is directed against said NS2- or NS3-selective antibody.

22. The method according to claim 16, wherein said K147 antibody is detected by using an anti-rabbit antibody that is linked to a detectable label.

23. A method for detecting NS3 product in a sample containing NS2/3 protease in the presence of about 1% LDAO, comprising:
a) diluting said sample in the presence of an activation agent to allow at least a portion of the NS2/3 protease to self-cleave to yield cleaved NS2 and NS3 products;
a′) further diluting said sample to stop auto-cleavage;
a″) immobilizing the NS3 product from step a′);
b) incubating the immobilized NS3 product of step a″) with a NS3-selective antibody which has no or low cross-reactivity with NS2 cleaved product and uncleaved NS2/3;
c) detecting the immobilized NS3-bound antibody produced in step b), whereby the amount of antibody detected correlates with the NS2/3 autocleavage activity.

24. The method according to claim 23, wherein step a′) and a″) can be performed in sequence, or in inverse sequence, or together in the same step, and wherein said dilution of said sample in step a′) can be carried out by washing said immobilized sample.

25. The method of claim 23, wherein said antibody is directed against a peptide comprising amino acid sequence: APITAYSQQT [SEQ ID NO.5].

26. The method of claim 25, wherein said antibody is labeled with europium.

27. The method of claim 25, wherein said antibody is K147 rabbit polyclonal antibody and is detected with the use of an anti-rabbit antibody that is labeled with europium.

28. An assay for screening a candidate compound for NS2/3 cleavage inhibitory activity in a sample containing NS2/3 protease, the assay comprising:
a) subjecting a first sample comprising NS2/3 protease to suitable conditions under which at least a portion of NS2/3 is self-cleaved to yield cleaved NS2 and NS3 products in the absence of a candidate compound;
b) subjecting a second sample comprising NS2/3 protease in the presence of a candidate compound under the same conditions as those in step a);
c) incubating the first and second samples with a ligand that preferentially binds to one of either NS2 or NS3 product over NS2/3 under conditions suitable to cause binding of the ligand to NS2 or NS3; and
d) determining the amount of ligand bound to the second sample and to the first sample produced in step c), whereby a decrease in the amount of ligand in the second sample compared to that of the first sample indicates that the candidate compound may be an inhibitor of NS2/3 autocleavage activity.

29. The assay according to claim 28, wherein LDAO is initially present in said NS2/3 sample to prevent autocleavage.

30. The assay according to claim 29, wherein said LDAO is present at a concentration of about 1%.

31. The assay according to claim 28, wherein in step a) said sample is diluted such that LDAO concentration is decreased at least about 1.5 fold and is incubated in the presence of an activation agent to induce autocleavage.

32. The assay according to claim 31, wherein said activation agent is a detergent selected from the group consisting of: CHAPS, Triton X-100, NP-40 and n-dodecyl-D-maltoside (DM).

33. The assay according to claim 32, wherein said detergent acting as activation agent is present above its CMC.

34. The assay according to claim 33, wherein in step a), said protease is incubated in a solution further comprising glycerol to enhance autocleavage.

35. The assay according to claim 34, wherein said glycerol is present at a concentration of about 20%.

36. The assay according to claim 28, wherein in step b) said ligand is an antibody.

37. The assay according to claim 36, wherein said antibody is a polyclonal antibody or a monoclonal antibody.

38. The assay according to claim 37, wherein said antibody is an antibody directed to amino acid sequences specific to NS2 cleaved product and said antibody is selective inasmuch as it has no or low cross-reactivity with NS3 cleaved product and uncleaved NS2/3.

39. The assay according to claim 38, wherein said NS2-selective antibody is an antibody directed against a peptide comprising an amino acid sequence selected from the group consisting of:
[SEQ ID NO. 6]
Ser-Phe-Glu-Gly-Gln-Gly-Trp-Arg-Leu-Leu;
[SEQ ID NO. 8]
Asn-Phe-Glu-Gly-Gln-Gly-Trp-Arg-Leu-Leu;
[SEQ ID NO. 9]
Asp-Asn-Phe-Glu-Gly-Gln-Gly-Trp-Arg-Leu;
[SEQ ID NO. 10]
Ala-Asp-Asn-Phe-Glu-Gly-Gln-Gly-Trp-Arg;
[SEQ ID NO. 11]
Pro-Ala-Asp-Asn-Phe-Glu-Gly-Gln-Gly-Trp;
[SEQ ID NO. 12]
Gly-Pro-Ala-Asp-Asn-Phe-Glu-Gly-Gln-Gly;
and
[SEQ ID NO. 13]
Ser-Ala-Arg-Arg-Gly-Arg-Glu-Ile-Leu-Leu.

40. The assay according to claim 37, wherein said antibody is an antibody directed to amino acid sequences specific to NS3 cleaved product and said antibody is selective inasmuch as it has no or low cross-reactivity with NS2 cleaved product and uncleaved NS2/3.

41. The assay according to claim 40, wherein said NS3-selective antibody is an antibody directed against a peptide comprising an amino acid sequence selected from the group consisting of:
[SEQ ID NO. 5]
Ala-Pro-Ile-Thr-Ala-Tyr-Ser-Gln-Gln-Thr;
[SEQ ID NO. 14]
Pro-Ile-Thr-Ala-Tyr-Ser-Gln-Gln-Thr-Thr;
[SEQ ID NO. 15]
Ile-Thr-Ala-Tyr-Ser-Gln-Gln-Thr-Thr-Arg;
[SEQ ID NO. 16]
Thr-Ala-Tyr-Ser-Gln-Gln-Thr-Thr-Arg-Gly;
and
[SEQ ID NO. 17]
Arg-Gly-Leu-Leu-Gly-Cys-Ile-Ile-Thr-Ser.

42. The assay according to claim 41, wherein said antibody is directed against a peptide comprising amino acid sequence: APITAYSQQT [SEQ ID NO.5].

43. The assay according to claim 42, wherein said antibody is K147 rabbit polyclonal antibody.

44. The assay according to claim 36, wherein said antibody is detected with the use of a detectable label.

45. The assay according to claim 44, wherein said detectable label is selected from the group consisting of: fluorescent label, chemiluminescent label, calorimetric label, enzymatic marker, and radioactive isotope.

46. The assay according to claim 45, wherein said detectable label is europium.

47. The assay according to claim 44, wherein said detectable label is linked directly to said antibody.

48. The assay according to claim 44, wherein said detectable label is linked to an antibody that is directed against said NS2- or NS3-selective antibody.

49. The assay according to claim 43, wherein said K147 antibody is detected by using an anti-rabbit antibody that is linked to a detectable label.

50. An assay for screening a candidate compound for NS2/3 cleavage inhibitory activity in a sample containing NS2/3 protease in the presence of about 1% LDAO, the assay comprising:
a) diluting a first sample comprising NS2/3 protease in the presence of an activation agent to allow at least a portion of NS2/3 is cleaved to yield cleaved NS2 and NS3 products, in the absence of candidate compound;
a′) further diluting the protease in said first sample to stop auto-cleavage;
a″) immobilizing the NS3 product from step a′);
b) subjecting a second sample comprising NS2/3 protease in the presence of a candidate compound under the same conditions as those in step a);
b′) diluting the protease in said second sample to achieve the same conditions as those in step a′);
b″) immobilizing the NS3 product, if any, from step b′);
c) incubating immobilized NS3 from step a″) with a NS3-selective antibody directly or indirectly labeled with a detectable marker, wherein said antibody preferentially binds to NS3 over NS2/3;
c′) incubating immobilized NS3 from step b″) with the same antibody as in step c);
d) determining the amount of labeled-antibody immobilized in each of step c) and c′), whereby a decrease in the amount of immobilized labeled-antibody in the step c′) as compared to that for step c) indicates that the candidate compound may be an inhibitor of NS2/3 cleavage activity.

51. The assay according to claim 50, wherein step a′) and a″) can be performed in sequence, or in inverse sequence, or together in the same step, and wherein said dilution of said sample in step a′) can be carried out by washing said immobilized sample.

52. The assay of claim 50, wherein said antibody is directed against the peptide APITAYSQQT [SEQ ID NO.5].

53. The assay of claim 52, wherein said antibody is labeled with europium.

54. The assay of claim 52, wherein said antibody is K147 rabbit polyclonal antibody and is detected with the use of an anti-rabbit antibody that is labeled with europium.

55. An antibody directed to amino acid sequences selective for NS3 cleaved product wherein said antibody has no or low cross-reactivity with NS2 cleaved product and uncleaved NS2/3.

56. The antibody according to claim 55, wherein said antibody yields a signal that is at least about 2 fold higher against NS3 cleaved product than against NS2 product and uncleaved NS2/3.

57. The antibody according to claim 55, wherein said NS3-selective antibody is an antibody directed against a peptide comprising an amino acid sequence selected from the group consisting of:
[SEQ ID NO. 5]
Ala-Pro-Ile-Thr-Ala-Tyr-Ser-Gln-Gln-Thr;
[SEQ ID NO. 14]
Pro-Ile-Thr-Ala-Tyr-Ser-Gln-Gln-Thr-Thr;
[SEQ ID NO. 15]
Ile-Thr-Ala-Tyr-Ser-Gln-Gln-Thr-Thr-Arg;
[SEQ ID NO. 16]
Thr-Ala-Tyr-Ser-Gln-Gln-Thr-Thr-Arg-Gly;
and
[SEQ ID NO. 17]
Arg-Gly-Leu-Leu-Gly-Cys-Ile-Ile-Thr-Ser.

58. The method according to claim 57, wherein said antibody is directed against the peptide APITAYSQQT [SEQ ID NO.5].

59. The method according to claim 58, wherein said antibody is the K147 polyclonal antibody.

60. An antibody directed to amino acid sequences selective for NS2 cleaved product wherein said antibody has no or low cross-reactivity with NS3 cleaved product and uncleaved NS2/3.

61. The antibody according to claim 60, wherein said antibody yields a signal that is at least about 2 fold higher against NS2 cleaved product than against NS3 product and uncleaved NS2/3.

62. The antibody according to claim 60, wherein said NS2-selective antibody is an antibody directed against a peptide comprising an amino acid sequence selected from the group consisting of:
[SEQ ID NO. 6]
Ser-Phe-Glu-Gly-Gln-Gly-Trp-Arg-Leu-Leu;
[SEQ ID NO. 8]
Asn-Phe-Glu-Gly-Gln-Gly-Trp-Arg-Leu-Leu;
[SEQ ID NO. 9]
Asp-Asn-Phe-Glu-Gly-Gln-Gly-Trp-Arg-Leu;
[SEQ ID NO. 10]
Ala-Asp-Asn-Phe-Glu-Gly-Gln-Gly-Trp-Arg;
[SEQ ID NO. 11]
Pro-Ala-Asp-Asn-Phe-Glu-Gly-Gln-Gly-Trp;
[SEQ ID NO. 12]
Gly-Pro-Ala-Asp-Asn-Phe-Glu-Gly-Gln-Gly;
and
[SEQ ID NO. 13]
Ser-Ala-Arg-Arg-Gly-Arg-Glu-Ile-Leu-Leu.


SEQUENCE LISTING (17 sequences)

SEQ ID NO: 1 - DNA, 1191 bp, Hepatitis C Virus
  CDS (1)...(1191): Nucleotide sequence of NS2/3
atggaccgggagatggctgcatcgtgcggaggcgcggttttcataggtcttgcactcttgaccttgtcaccatactataaagtgctcctcgctaggctcatatggtggttacagtatttaatcaccagagtcgaggcgcacttgcaagtgtggatcccccctctcaatgttcggggaggccgcgatgccatcatcctcctcacgtgcgcagtccacccagagctaatctttgacatcaccaaactcctgctcgccatattcggtccgctcatggtgctccaggcaggcataaccaaagtgccgtacttcgtgcgtgcgcaggggctcattcgtgcgtgtatgttggtgcggaaggctgcggggggtcattatgtccaaatggccttcatgaagctagctgcgctgacaggtacgtacgtttatgaccatctcactccattgcaggattgggcccacgcgggcctacgagaccttgcagtggcggtagagcccgtcatcttctctgacatggaggtcaagatcatcacctggggggcggacaccgcggcatgcggggacatcatttcaggtctgcccgtctccgctcgaaggggaagggagatactcctgggaccggccgataattttgaagggcaggggtggcgactccttgcgcccatcacggcctactcccaacagacacggggcctacttggttgcatcatcaccagcctcacaggccgggacaagaaccaggtcgagggggaggttcaagtggtctccaccgctacacaatctttcctggcgacctgcgtcaacggcgtgtgttggactgtcttccatggcgccggctcaaagaccttggccggccccaaaggcccaatcacccagatgtacactaatgtggaccaggacctcgtcggctggcaggcgccccctggggcgcgctccatgacaccatgcacctgcggcagctcggacctctatttggtcacgagacatgccgacgtcattccggtgcgccggcggggcgacagtagggggagcctgctctcccccaggcctgtctcctacttgaagggctcttcgggtggcccactgctctgcccttcggggcacgttgtgggcatcttccgggctgctgtgtgcacccggggggttgcaaaagcggtggacttcatacctgttgagtctatggaaactaccatgcgg

SEQ ID NO: 2 - protein, 300 aa, Hepatitis C Virus
TKVPYFVRAQGLIRACMLVRKAAGGHYVQMAFMKLAALTGTYVYDHLTPLQDWAHAGLRDLAVAVEPVIFSDMEVKIITWGADTAACGDIISGLPVSARRGREILLGPADNFEGQGWRLLAPITAYSQQTRGLLGCIITSLTGRDKNQVEGEVQVVSTATQSFLATCVNGVCWTVFHGAGSKTLAGPKGPITQMYTNVDQDLVGWQAPPGARSMTPCTCGSSDLYLVTRHADVIPVRRRGDSRGSLLSPRPVSYLKGSSGGPLLCPSGHVVGIFRAAVCTRGVAKAVDFIPVESMETTMR

SEQ ID NO: 3 - protein, 303 aa, Hepatitis C Virus
AGITKVPYFVRAQGLIRACMLVRKAAGGHYVQMAFMKLAALTGTYVYDHLTPLQDWAHAGLRDLAVAVEPVIFSDMEVKIITWGADTAACGDIISGLPVSARRGREILLGPADNFEGQGWRLLAPITAYSQQTRGLLGCIITSLTGRDKNQVEGEVQVVSTATQSFLATCVNGVCWTVFHGAGSKTLAGPKGPITQMYTNVDQDLVGWQAPPGARSMTPCTCGSSDLYLVTRHADVIPVRRRGDSRGSLLSPRPVSYLKGSSGGPLLCPSGHVVGIFRAAVCTRGVAKAVDFIPVESMETTMR

SEQ ID NO: 4 - protein, 334 aa, Hepatitis C Virus
MKKKKLEHHHHHHTSAGITKVPYFVRAQGLIRACMLVRKAAGGHYVQMAFMKLAALTGTYVYDHLTPLQDWAHAGLRDLAVAVEPVIFSDMEVKIITWGADTAACGDIISGLPVSARRGREILLGPADNFEGQGWRLLAPITAYSQQTRGLLGCIITSLTGRDKNQVEGEVQVVSTATQSFLATCVNGVCWTVFHGAGSKTLAGPKGPITQMYTNVDQDLVGWQAPPGARSMTPCTCGSSDLYLVTRHADVIPVRRRGDSRGSLLSPRPVSYLKGSSGGPLLCPSGHVVGIFRAAVCTRGVAKAVDFIPVESMETTMRTSSAWRHPQFGGKKKK

SEQ ID NO: 5 - protein, 10 aa, Hepatitis C Virus
APITAYSQQT

SEQ ID NO: 6 - protein, 10 aa, Hepatitis C Virus
SFEGQGWRLL

SEQ ID NO: 7 - protein, 17 aa, Hepatitis C Virus
KKGSVVIVGRIILSGRK

SEQ ID NO: 8 - protein, 10 aa, Hepatitis C Virus
NFEGQGWRLL

SEQ ID NO: 9 - protein, 10 aa, Hepatitis C Virus
DNFEGQGWRL

SEQ ID NO: 10 - protein, 10 aa, Hepatitis C Virus
ADNFEGQGWR

SEQ ID NO: 11 - protein, 10 aa, Hepatitis C Virus
PADNFEGQGW

SEQ ID NO: 12 - protein, 10 aa, Hepatitis C Virus
GPADNFEGQG

SEQ ID NO: 13 - protein, 10 aa, Hepatitis C Virus
SARRGREILL

SEQ ID NO: 14 - protein, 10 aa, Hepatitis C Virus
PITAYSQQTT

SEQ ID NO: 15 - protein, 10 aa, Hepatitis C Virus
ITAYSQQTTR

SEQ ID NO: 16 - protein, 10 aa, Hepatitis C Virus
TAYSQQTTRG

SEQ ID NO: 17 - protein, 10 aa, Hepatitis C Virus
RGLLGCIITS